(19) United States
(12) Patent Application Publication
(10) Pub. No.: US 2007/0255227 A1
(43) Pub. Date: Nov. 1, 2007

(54) Methods and apparatus for refilling an infusion device

(76) Inventor: James Martin Haase, Maplewood, MN (US)

Correspondence Address: MUETING, RAASCH & GEBHARDT, P.A., P.O. BOX 581415, MINNEAPOLIS, MN, 55458, US

(21) Appl. No.: 11/412,443

(22) Filed: Apr. 27, 2006

(51) Int. Cl. A61M 5/315

(52) U.S. Cl. 604/218

(57) ABSTRACT

Apparatus and method for use in aspirating and/or refilling a reservoir of an implantable infusion device. In one embodiment, the apparatus includes one or more syringes and a plunger stop for holding a plunger of the syringe(s) in a minimally retracted position relative to a barrel of the syringe(s). The apparatus may further include a filter attachable to a syringe, wherein the filter permits the delivery of a therapeutic substance contained in the syringe while blocking the passage of gas bubbles within the therapeutic substance. Methods of aspirating and refilling a reservoir of the infusion device are also provided.

### TECHNICAL FIELD

The present invention relates generally to medical devices and, more particularly, to apparatus, systems, and methods for refilling a reservoir of an infusion pump.

### BACKGROUND

Treatment of diseases and ailments of the body often benefit from short- or long-term infusion of drugs and/or other fluids. While such therapeutic substances may be administered extracorporeally, e.g., via transcutaneous injection, many patient benefit from the consistent and repeatable dosage provided by an implantable infusion pump. Such pumps may be used in a variety of applications such as control of pain and/or spasticity. They are well-suited to deliver infusate fluids to a targeted delivery site such as an epidural or intrathecal space of the spinal canal, or a particular location within the brain.

Implantable infusion pumps are typically implanted subcutaneously, e.g., in the chest or abdominal cavity. The pump may incorporate a reservoir to hold the infusate fluid. A self-sealing, needle-penetrable septum may also be provided and is preferably located generally directly beneath the skin. The septum provides a fluid passageway that permits the reservoir to be refilled periodically via a transcutaneous injection. Accordingly, the pump reservoir can be filled or refilled without requiring surgical removal from the patient's body, and further without requiring any other significant surgical procedure.

The pump may also include a discharge outlet through which the therapeutic substance is directed during delivery. The outlet is typically connected to flexible medical tubing, e.g., a catheter, leading to the targeted delivery site. In addition to the reservoir, some infusion pumps may further include a power source, a pump, and associated electronics to control delivery of the therapeutic substance to the patient in accordance with a prescribed schedule.

One type of implantable infusion pump includes a reservoir that is subjected to a storage pressure that is less than ambient body pressure (e.g., subjected to a relative negative pressure). As a result, unintended leakage of the substance from the reservoir, which may occur with positive or neutral pressure devices, may be substantially reduced or prevented.

While implantable infusion pumps provide substantial benefits, refilling of the pump reservoir is sometimes perceived as a time-consuming and potentially difficult procedure, particularly with respect to negative pressure reservoir devices. For example, standard plunger-in-barrel syringes have been used in combination with appropriate transcutaneous needles to access the pump reservoir. In these configurations, plunger retraction or advancement may be used to, respectively, aspirate residual therapeutic substance from, or supply the substance to, the reservoir.

Unfortunately, it is sometimes difficult to hold the syringe securely while applying the necessary force needed to manipulate the syringe plunger. For example, it may be challenging to hold the syringe while also applying sufficient retraction force to the plunger during aspiration of the reservoir. As a result, refill procedures have often required a relatively high level of skill. Moreover, it is often necessary to degas the therapeutic substance prior to injecting it into the pump reservoir. Thus, some procedures have utilized relatively costly and sophisticated degassing equipment.

What is needed is a system and method that overcomes these and other problems associated with refilling a reservoir of an implantable infusion device.

### SUMMARY

The present invention provides a kit and method for delivering therapeutic substance to the reservoir of an implantable infusion pump. Systems and methods described herein may also provide effective means for degassing the substance prior to delivery to the reservoir. Embodiments of the present invention may also provide an aspiration or refill syringe adapted for drawing or delivering medication without requiring concurrent manipulation of a syringe plunger.

In one embodiment, a syringe for use in aspirating or delivering a therapeutic substance is provided. The syringe includes a barrel having a distal end for coupling with a fluid transport component, and an open proximal end. The syringe further includes an elongate plunger having a piston end slidably receivable within the open proximal end of the barrel, and a button end to impart motion to the plunger relative the barrel. An elongate plunger stop is also provided and coupled to the plunger at or near the button end. The plunger stop is selectively movable to an engaged position whereby the plunger stop extends along the plunger between the button end and the open proximal end of the barrel.

In another embodiment, a kit is provided for use in aspirating and/or refilling a negative pressure reservoir of an infusion device. The kit includes a syringe having: a barrel with a distal end and an open proximal end; and a plunger introducible via the open proximal end of the barrel and slidable therein. The kit further includes a tube for fluidly coupling the syringe to the infusion device, and a filter for fluid attachment between the syringe and the reservoir. A control valve positionable along the tube between the filter and the reservoir is also provided. The control valve is selectively movable between an open position, wherein the tube is open, and a closed position, wherein the tube is occluded. The kit also includes a plunger stop attachable to the plunger at or near a button end of the plunger, the plunger stop operable to selectively limit plunger movement relative to the barrel.

In yet another embodiment, a method for use in filling a reservoir of an infusion device with a therapeutic substance is provided. The method includes filling a syringe with a predetermined volume of the therapeutic substance, wherein the syringe includes: a barrel with a distal end and an open proximal end; and a plunger introducible via the open proximal end of the barrel and slidable therein. The method further includes: purging air from the syringe; attaching a filter to a discharge outlet located at the distal end of the barrel; purging air from the filter; retracting the plunger from the barrel and holding the plunger in a retracted position relative to the barrel; agitating the syringe with the predetermined volume of the therapeutic substance therein; and releasing the plunger from the retracted position. Still further, the method provides: attaching an outlet of the filter to a tube fluidly coupled to the reservoir of the infusion device; opening a control valve operatively coupled to the tube to permit flow through the tube; transferring the predetermined volume of the therapeutic substance from the syringe to the infusion device via vacuum pressure present within the reservoir; and blocking transfer of gas bubbles from the syringe to the reservoir with the filter.

In still another embodiment, a method for use in refilling a reservoir of an infusion device is provided. The method includes connecting an outlet of an aspiration syringe to a tube, wherein the aspiration syringe includes: a barrel with a distal end and an open proximal end; and a plunger introducible via the open proximal end of the barrel and slidable therein. The method further includes closing a tubing clamp operatively connected to the tube; connecting the tube to a needle and inserting the needle into a refill port of the infusion device; retracting the plunger of the aspiration syringe to draw vacuum pressure in the aspiration syringe; engaging a plunger stop associated with the aspiration syringe to hold the plunger of the aspiration syringe in a retracted position relative to the barrel of the aspiration syringe; opening the tubing clamp to draw residual therapeutic substance from the reservoir under the vacuum pressure created by the aspiration syringe; closing the tubing clamp; and disconnecting the aspiration syringe from the tube. The method further includes: preparing a refill syringe containing a predetermined volume of the therapeutic substance therein; purging air from the refill syringe; attaching a filter to a discharge outlet of the refill syringe; purging air from the filter; retracting a plunger of the refill syringe and engaging a plunger stop associated with the refill syringe to hold the plunger of the refill syringe in a retracted position relative to a barrel of the refill syringe; and agitating the refill syringe while the predetermined volume of the therapeutic substance resides therein. The method further includes: disengaging the plunger stop associated with the refill syringe so that the plunger of the refill syringe may move into the barrel of the refill syringe; attaching an outlet of the filter to the tube; opening the tubing clamp; and transferring the predetermined volume of the therapeutic substance from the refill syringe to the reservoir via vacuum pressure in the reservoir.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIG. 1 illustrates a refill kit in accordance with one embodiment of the invention;

FIGS. 2A-2C illustrate an exemplary plunger stop for use with a syringe, wherein: FIG. 2A is a partial perspective view of the syringe with the plunger stop shown in a disengaged or unlocked position; FIG. 2B is a partial perspective view of the syringe and plunger stop of FIG. 2A with the plunger stop shown in an engaged or stop position; and FIG. 2C is a partial top plan view of the plunger stop (with the syringe removed for clarity);

FIG. 3 is a diagrammatic view of an implantable infusion device refillable with the kit of FIG. 1; and

FIG. 4 is a flow diagram illustrating a process for refilling a reservoir of an implantable infusion pump with a therapeutic substance, wherein the diagram further illustrates an optional method for aspirating residual substance from the reservoir prior to refilling.

The figures are rendered primarily for clarity and, as a result, are not necessarily drawn to scale.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the invention.

The present invention is directed to medical devices and, in particular, to apparatus, systems, and methods for refilling infusion devices, e.g., implantable infusion devices, with a therapeutic substance. In one embodiment of the invention, a kit is provided that may be used to assist a clinician in aspirating and/or refilling a reservoir of the infusion device. A stop mechanism, e.g., plunger stop, capable of maintaining a plunger of a syringe in a minimally retracted position may also be provided.

It is noted that the terms “comprises” and variations thereof do not have a limiting meaning where these terms appear in the accompanying description and claims. Moreover, “a,” “an,” “the,” “at least one,” and “one or more” are used interchangeably herein.

Relative terms such as left, right, forward, rearward, top, bottom, side, upper, lower, horizontal, vertical, and the like may be used herein and, if so, are from the perspective observed in the particular figure. These terms are used only to simplify the description, however, and not to limit the scope of the invention in any way.

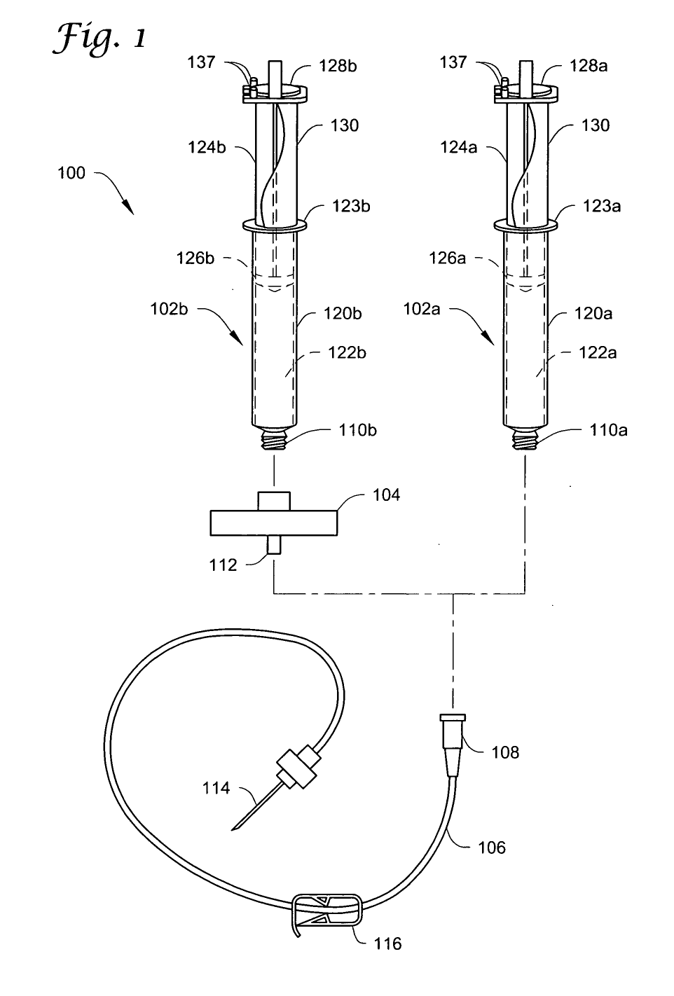

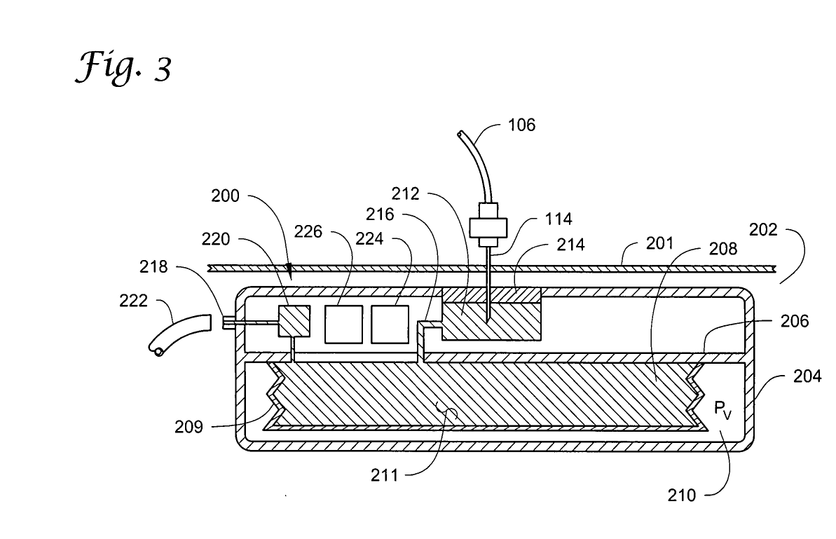

FIG. 1 depicts an exemplary filling/refilling system or kit 100 in accordance with one embodiment of the invention. The system 100 may be used to refill a medical device (such as an implantable infusion device 200 illustrated in FIG. 3) as further described below. The system 100 may include at least one syringe 102. In the illustrated embodiment, a first or aspirating syringe 102a is provided to aspirate residual substance from the device reservoir, while a second or refill syringe 102b is provided to replenish the reservoir. The system 100 may further include a filter 104 and a filling tube 106 (the filling tube is also referred to herein as “tubing”). The filling tube 106 may include a connector 108 at its proximal end that permits fluid coupling of the tube to a discharge outlet 110 of either syringe 102a or 102b or a discharge outlet 112 of the filter 104. A distal end of the tube 106 may permit fluid connection with the infusion device 200. The connector 108 may form a luer lock connection that allows it to be connected quickly and easily to the filter 104 or to the syringes 102.

The syringes 102a and 102b are preferably, but not necessarily, identical.

The reference numeral suffixes “a” and “b” are used herein to denote substantially similar parts or features of the two illustrated syringes. Unless otherwise identified herein, the description of an individual syringe (e.g., syringe 102a) also applies to the corresponding syringe (e.g., syringe 102b). Similarly, unless otherwise noted, the description of a syringe feature/component identified without a suffix applies to each syringe (e.g., syringes 102a and 102b).

In one embodiment, the filter 104, which may be fluidly attached between the syringe and reservoir, is a 0.22 micron bacterial filter identified by model no. SLGV 025 N, produced by Millipore Corp. of Billerica, Mass., USA. However, other filters may certainly be used without departing from the scope of the invention.

The system 100 may further include a needle 114 that is attachable to, or preassembled with, a distal end of the filling tube 106. The needle 114 is operable to penetrate skin of the patient and enter the infusion device as further described below. A control valve, e.g., a tubing clamp 116, may also be provided. The tubing clamp 116, which may be positioned along the tube 106 between the syringe 102 (or the filter 104) and the reservoir, is operable to move between an open position (wherein the tube is open to permit flow), and a closed position (wherein the tube is closed or occluded such that flow is terminated).

While illustrated as a mechanical tube compression device, the tubing clamp 116 may be of most any configuration that permits selective opening and closing of the tube 106. For example, although not shown, the tubing clamp 116 could be configured as a petcock-type valve.

Each syringe 102 preferably includes a barrel 120 defining an interior substance chamber 122. A distal end of the barrel is closed except for a small passageway defining the discharge outlet 110 such that the distal end may be coupled with a fluid transport component, e.g., the tube 106. The opposite end of the barrel 120 defines an open proximal end preferably having a flange 123 formed thereon. Each syringe 102 may further include an elongate plunger 124 having a piston or piston end 126 that is introducible and slidably received within the open proximal end of the barrel 120. When positioned within the barrel 120, a seal located at the piston end 126 forms a generally liquid and airtight seal with the barrel. A second end of the plunger 124 may define a button or button end 128 used to impart motion to the plunger relative to the barrel 120. By sliding the plunger 124 relative to the barrel 120, e.g., by relative pulling or pushing of the button end 128 relative to the barrel 120, the volume of the substance chamber 122 may be increased or decreased, respectively.

The system 100 (e.g., syringes 102) may further include a plunger stop 130 in accordance with one embodiment of the invention. While each syringe 102 is shown as having a separate plunger stop 130, other embodiments of the invention may provide a single plunger stop that may be attached/detached from different syringes as desired.

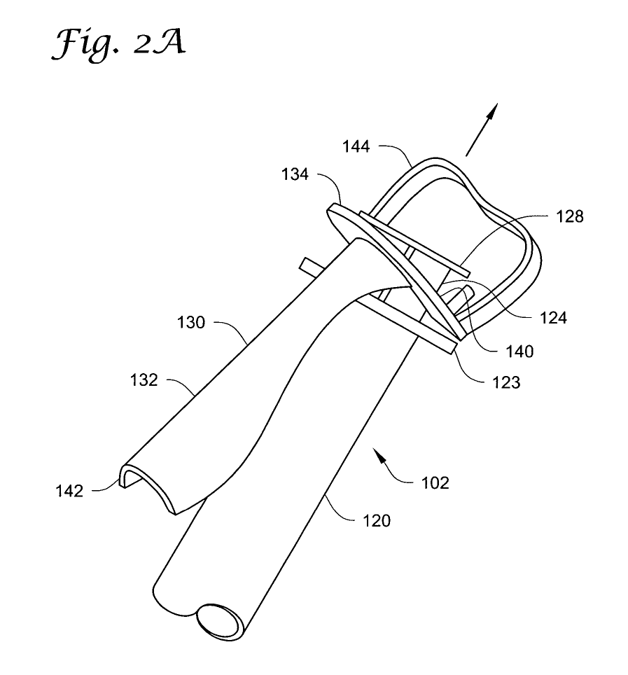

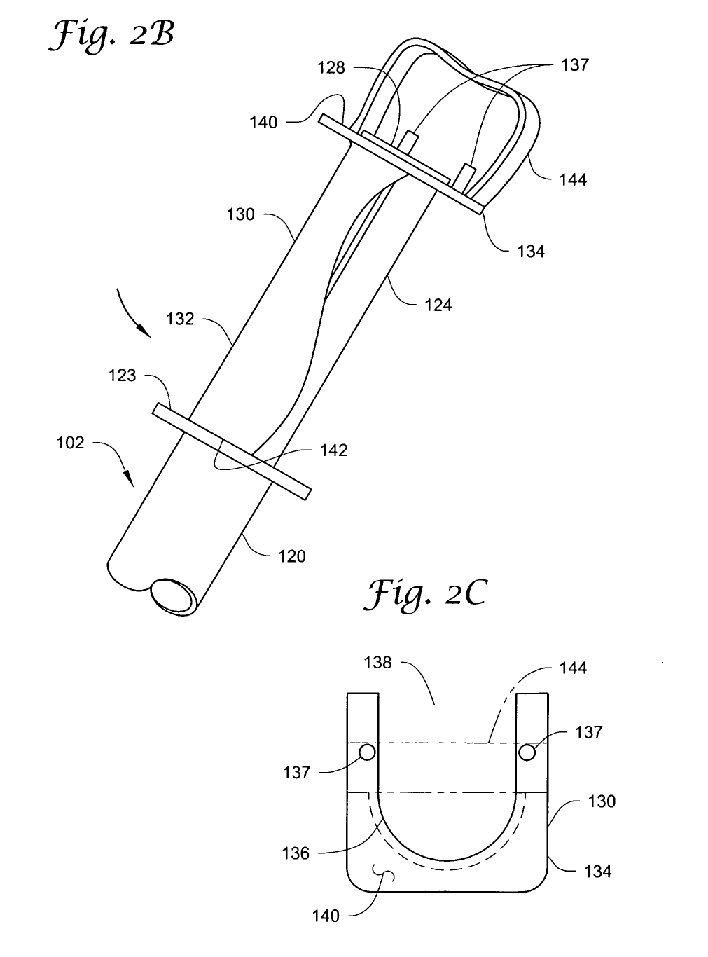

FIGS. 2A-2C illustrate the exemplary plunger stop 130 in greater detail. FIG. 2A is a perspective view of the plunger stop 130 as it is coupled, e.g., pivotally coupled, to the plunger 124 at or near the button end 128 while the plunger is in a fully depressed position relative to the barrel 120. In other words, the plunger stop 130 is shown in a disengaged or unlocked position. FIG. 2B, on the other hand, illustrates the plunger stop 130 after it is moved to an engaged or stop position. In the stop position, the plunger stop 130 selectively limits movement of the plunger 124 relative to the barrel 120, e.g., it limits further movement of the plunger into the barrel by holding the plunger in a minimally retracted position as shown. As illustrated in the Figures, the plunger stop 130 may be configured to permit further retraction of the plunger from the barrel 120 while in the stop position. FIG. 2C is a top plan view of the plunger stop 130.

The exemplary plunger stop 130 illustrated in the figures includes a generally semi-cylindrical body 132 and a head 134. The head 134 may define an opening 136 (see FIG. 2C) and a slot 138 that extends from the opening through a peripheral edge of the head. Preferably, a clearance between the diameter of the opening 136 and a diameter of the plunger 124 is sufficient to permit selective pivoting of the plunger stop about the plunger, e.g., between the stop position (FIG. 2B) and the unlocked position (FIG. 2A).

The head 134 may also include features that assist in maintaining the plunger stop in place during operation. For example, two posts 137 may be provided to engage the button end 128 of the plunger 124 as shown in FIG. 1. The posts 137 may reduce or prevent sideways movement of the plunger stop 130 relative to the plunger during syringe use.

When the plunger stop 130 is in the stop position of FIG. 2B, a first surface 140 of the plunger stop 130 may engage a corresponding surface of the button end 128, while a second surface 142 may engage the flange 123 of the barrel 120. In the stop position, the plunger stop 130 extends along the plunger 124 between the button end and the open proximal end of the barrel 120. As a result, the plunger stop 130 may prevent the movement of the plunger 124 from moving further into the barrel, e.g., the plunger stop 130 may be used to maintain the volume of the chamber 122 (see FIG. 1) at or above a predetermined threshold.

The plunger stop 130 may also be moved, e.g., pivoted, to the unlocked position as shown in FIG. 2A. In this position, the plunger stop does not interfere with the motion of the plunger 124 relative to the barrel 120. That is, in the stop position, the distal end of the plunger stop 130 is positioned proximate the plunger 124 (see FIG. 2B); while, in the unlocked position, the distal end of the plunger stop is positioned away from both the plunger and the barrel 120 (see FIG. 2A). To assist the clinician in retraction of the plunger 124 relative to the barrel 120, the head 134 may also include a finger loop 144.

In one embodiment, the finger loop 144 is positioned off of the centerline of the plunger stop as shown in FIG. 2C. Thus, application of a pulling force at the finger loop 144 (in the direction indicated by the arrow in FIG. 2A) tends to bias the second end 142 of the plunger stop inwardly towards the plunger 124. Stated alternatively, the plunger stop 130 tends to move into the desired stop position of FIG. 2B once the plunger 124 has been adequately withdrawn from the barrel 120 using the finger loop 144.

The kit 100 is operable to assist the clinician in aspirating and/or refilling the infusion device 200, which is represented diagrammatically in FIG. 3. The infusion device 200 may be implanted close to the skin 201 of the patient's body 202. Although the specific operation of the device 200 is not central to the invention, it is briefly described below. The device 200 may include a housing 204 with a bulkhead 206 that divides the interior of the housing into two or more chambers. A reservoir 208 that, in the illustrated embodiment, may be formed by a collapsible bellows 209, is provided and sealed against a lower side of the bulkhead 206. The reservoir 208 may hold the therapeutic substance 211 therein. A propellant chamber 210 surrounds the reservoir 208. In the illustrated embodiment, the propellant chamber is subject to vacuum (e.g., negative gage) pressure Pv.

The housing 204 of the device 200 may include an inlet port 212 through which the needle 114 of the kit 100 may enter to aspirate/refill the reservoir 208. The inlet port may include a self-sealing, needle-penetrable septum 214 as is known in the art. The inlet port may also be coupled to the reservoir 208 via a refill passageway 216. In addition to the inlet port, the housing 204 may include an outlet port 218 for delivering the therapeutic substance 211 to the patient. The therapeutic substance may be transferred from the reservoir 208 to the outlet port 218 via a pumping mechanism 220, e.g., a piston pump or peristaltic pump. A catheter 222 may deliver the therapeutic substance from the outlet port 218 to the area of the body 202 intended to receive medication. The device 200 may include other components, e.g., a power source 224 and controls 226, as is known in the art.

During the refill process, it is advantageous to aspirate the reservoir 208 of residual therapeutic substance to reduce the potential for gas formation. A kit like the kit 100 described above may assist with the refilling and/or aspiration of the reservoir 208 as further described below.

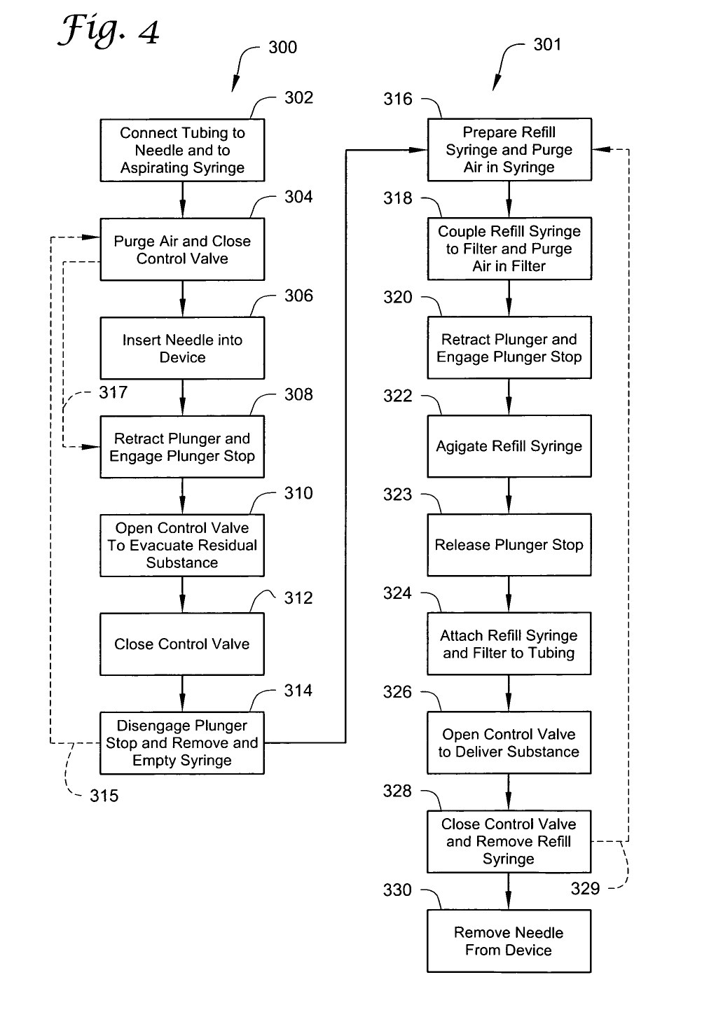

FIG. 4 illustrates an exemplary process for first aspirating residual substance from the reservoir 208 (process 300), and then refilling the reservoir with a predetermined volume of the therapeutic substance (process 301). In the following description, reference is made to the features/components of the kit 100 and the device 200 already described and illustrated herein. While both aspiration and refilling are shown in FIG. 4, it is contemplated that separate kits could be produced to accomplish each of these processes without departing from the scope of the invention.

To aspirate the reservoir 208, the tube 106 (see FIG. 1) may be connected, at one end, to the needle 114 and, at the other end, to the outlet of an aspiration syringe (e.g., syringe 102a of FIG. 1) as indicated at 302 in FIG. 4. It is then desirable to purge air from the syringe 102a (e.g., by pushing the plunger 124 completely into the barrel 120 (see FIG. 2A)) and then closing the control valve (e.g., tubing clamp 116) to occlude the tube as represented at 304. It may be beneficial to place the tubing clamp 116 close to the syringe 102 prior to closing the valve. The needle 114 may then be inserted through the septum 214 (see FIG. 3) and into the device 200 as shown at 306.

At this point, the plunger 124 may be retracted to draw a vacuum and the plunger stop 130 placed in the engaged or stop position illustrated in FIG. 2B as represented at 308. The plunger stop 130 may be retracted by application of a separating force between the barrel 120 and the button end 128. Advantageously, the force may be applied at the finger loop 144 such that when the plunger 124 is sufficiently retracted, the plunger stop 130 may be biased automatically towards the stop position illustrated in FIG. 2B. With the plunger stop 130 in place, vacuum pressure is applied between the syringe and the tubing clamp 116.

The tubing clamp 116 may be opened at 310, thereby applying a vacuum to the reservoir 208. The vacuum is preferably sufficient to overcome the negative pressure Pv in the propellant chamber 210 (see FIG. 3). As a result, the residual substance contained in the reservoir 208 may be drawn into the syringe 102a. At the cessation of bubbles entering the syringe 102a (it may be helpful to hold the syringe with the tube 106 extending downwardly to better observe any gas bubbles), the tubing clamp 116 may be closed at 312 and the plunger stop 130 moved to the disengaged or unlocked position. The syringe 102a may then be removed from the tube 106 and emptied at 314. If the volume of substance 211 in the syringe 102a is above a predetermined threshold (e.g., greater than about 20 milliliters (ml) in a 30 ml syringe), activities 304, 308, 310, 312, and 314 may be repeated as represented at 315 and 317.

After the reservoir 208 is aspirated, refilling may begin. In the process embodiment 301 illustrated in FIG. 4, a refill syringe, e.g., syringe 102b of FIG. 1, may be prepared by filling it with a predetermined volume of the therapeutic substance 211 at 316. Any air may be purged from the syringe 102b after filling by, for example, holding the syringe with the discharge outlet 110b pointing upwardly (and thus the plunger pointing downwardly) and advancing the plunger 124. The discharge outlet 110b of the syringe 102b (of the barrel 120) may then be attached or coupled to an inlet port of the filter 104 after which any air in the filter may be purged as represented at 318. Purging of air in the filter 104 may similarly be accomplished by holding the syringe with the filter 104 above the syringe (with the plunger 124 pointing downwardly) and advancing the plunger. This may also wet the filter 104 with the therapeutic substance 211. The plunger 124 may then be retracted from the barrel 120 and the plunger stop 130 engaged at 320 in a manner similar to that already described above with respect to the aspirating sequence 300, thereby holding the plunger in the retracted position (see FIGS. 1 and 2B).

The syringe 102b may then be agitated with the predetermined volume of therapeutic substance 211 therein for a period of time, e.g., about 10 second to about 20 seconds, as represented at 322. In its simplest form, agitation merely requires shaking the syringe/filter until the substance contained therein becomes cloudy with gas bubbles. The plunger stop 130 may then be released, e.g., moved to the unlocked position (see FIG. 2A), as shown at 323. The discharge outlet 112 of the filter 104 may then be attached to the connector 108 of the tube 106 (with the tubing clamp 116 closed). At this point, the tubing clamp 116 may be opened to allow flow of the substance to the reservoir 208 due to the reservoir's vacuum pressure as shown at 326. If desired, the transfer of substance to the reservoir may be accelerated by depressing the plunger 124.

The filter 104 preferably includes a substantially hydrophilic material so that it permits the passage of fluid, e.g., substance 211, through the filter while restricting, e.g., substantially blocking, transfer of gas bubbles from the syringe. That is, gas bubble passage from the syringe to the reservoir is substantially reduced or eliminated. Exemplary filter materials may include 0.22 micron pore size hydrophilic polyvinylidene fluoride (PVDF) and 0.2 micron pore size hydrophilized polytetrafluoroethylene (PTFE).

The filter is preferably selected to have a pore size small enough to provide a bubble point pressure (the minimum pressure required to force an air bubble through the wetted filter) higher than about 1 bar. For example, a 0.22 micron pore size PVDF filter may have a bubble point pressure of about 3.4 bar, while the 0.2 micron hydrophilized PTFE filter may have a bubble point pressure of about 13.6 bar. Filters having smaller pore sizes (e.g., about 0.1 micron) may be equally or more effective.

Once the substance is delivered from the syringe 102b, the tubing clamp 116 may be closed and the syringe/filter removed from the connector 108 as shown at 328. If additional substance is required to fill the reservoir 208, activities 316, 318, 320, 322, 323, 324, 326, and 328 may be repeated as indicated at 329. Optionally, another refill syringe 102b and filter 104, which may be provided with the kit 100, may be utilized if a second filling process is undertaken. When the reservoir is filled, the needle 114 may be removed from the device 200 as shown at 330.

Systems and methods pertaining to aspiration and/or refilling of an implantable infusion device are provided herein. In one embodiment, a syringe plunger stop is provided that permits application of aspiration vacuum pressure to the syringe without requiring the clinician to apply a concurrent withdrawal force to the plunger. Moreover, the introduction of a hydrophilic filter into the refill passageway may permit degassing the therapeutic substance within the syringe. It is contemplated that these and other features described herein may simplify the process of aspirating and/or refilling an infusion pump, particularly a negative pressure infusion pump.

Illustrative embodiments of this invention are discussed and reference has been made to possible variations within the scope of this invention. These and other variations, combinations, and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. A syringe for use in aspirating or delivering a therapeutic substance, the syringe comprising:
a barrel comprising a distal end for coupling with a fluid transport component, and an open proximal end;
an elongate plunger comprising a piston end slidably receivable within the open proximal end of the barrel, and a button end to impart motion to the plunger relative to the barrel; and
an elongate plunger stop coupled to the plunger at or near the button end, the plunger stop selectively movable to an engaged position whereby the plunger stop extends along the plunger between the button end and the open proximal end of the barrel.

2. The syringe of claim 1, wherein a first end of the plunger stop is pivotally attached at or near the button end of the plunger.

3. The syringe of claim 2, wherein the plunger stop is pivotable from the engaged position, wherein the distal end of the plunger stop is positioned proximate the plunger, to an unlocked position, wherein the distal end of the plunger stop is positioned away from the plunger.

4. The syringe of claim 1, wherein the plunger stop comprises first and second surfaces to engage, respectively, the button end of the plunger and a flange formed on the open proximal end of the barrel when the plunger stop is in the engaged position.

5. A kit for use in aspirating and/or refilling a negative pressure reservoir of an infusion device, the kit comprising:
a syringe comprising: a barrel with a distal end and an open proximal end; and a plunger introducible via the open proximal end of the barrel and slidable therein;
a tube for fluidly coupling the syringe to the infusion device;
a filter for fluid attachment between the syringe and the reservoir;
a control valve positionable along the tube between the filter and the reservoir, the control valve selectively movable between an open position, wherein the tube is open, and a closed position, wherein the tube is occluded; and
a plunger stop attachable to the plunger at or near a button end of the plunger, the plunger stop operable to selectively limit plunger movement relative to the barrel.

6. The kit of claim 5, further comprising a needle operable to pierce a self-sealing septum of the infusion device.

7. The kit of claim 5, wherein the control valve comprises a tubing clamp attachable to the tube.

8. The kit of claim 5, wherein the filter permits passage of fluid contained in the barrel while restricting passage of gas bubbles.

9. The kit of claim 5, wherein the filter comprises a substantially hydrophilic material.

10. The kit of claim 9, wherein the substantially hydrophilic material comprises polyvinylidene fluoride (PVDF) or polytetrafluoroethylene (PTFE).

11. The kit of claim 5, wherein the syringe is configurable to refill the reservoir, and wherein the kit comprises a second syringe configurable to aspirate the reservoir.

12. A method for use in filling a reservoir of an infusion device with a therapeutic substance, the method comprising:
filling a syringe with a predetermined volume of the therapeutic substance, the syringe comprising: a barrel with a distal end and an open proximal end; and a plunger introducible via the open proximal end of the barrel and slidable therein;
purging air from the syringe;
attaching a filter to a discharge outlet located at the distal end of the barrel;
purging air from the filter;
retracting the plunger from the barrel and holding the plunger in a retracted position relative to the barrel;
agitating the syringe with the predetermined volume of the therapeutic substance therein;
releasing the plunger from the retracted position;
attaching an outlet of the filter to a tube fluidly coupled to the reservoir of the infusion device;
opening a control valve operatively coupled to the tube to permit flow through the tube;
transferring the predetermined volume of the therapeutic substance from the syringe to the infusion device via vacuum pressure present within the reservoir; and
blocking transfer of gas bubbles from the syringe to the reservoir with the filter.

13. The method of claim 12, further comprising depressing the plunger to assist in transferring the therapeutic substance from the syringe to the infusion device.

14. The method of claim 12, wherein agitating the syringe comprises agitating the syringe until gas bubbles are visible therein.

15. The method of claim 12, further comprising aspirating the reservoir prior to transferring the predetermined volume of the therapeutic substance from the syringe.

16. The method of claim 12, wherein purging air from the syringe comprises holding the syringe with the plunger extending downwardly and advancing the plunger into the barrel.

17. The method of claim 12, wherein purging air from the filter comprises holding the syringe with the plunger extending downwardly and advancing the plunger into the barrel.

18. A method for use in refilling a reservoir of an infusion device, the method comprising:
connecting an outlet of an aspiration syringe to a tube, the aspiration syringe comprising: a barrel with a distal end and an open proximal end; and a plunger introducible via the open proximal end of the barrel and slidable therein;
closing a tubing clamp operatively connected to the tube;
connecting the tube to a needle and inserting the needle into a refill port of the infusion device;
retracting the plunger of the aspiration syringe to draw vacuum pressure in the aspiration syringe;
engaging a plunger stop associated with the aspiration syringe to hold the plunger of the aspiration syringe in a retracted position relative to the barrel of the aspiration syringe;
opening the tubing clamp to draw residual therapeutic substance from the reservoir under the vacuum pressure created by the aspiration syringe;
closing the tubing clamp;
disconnecting the aspiration syringe from the tube;
preparing a refill syringe containing a predetermined volume of the therapeutic substance therein;
purging air from the refill syringe;
attaching a filter to a discharge outlet of the refill syringe;
purging air from the filter;
retracting a plunger of the refill syringe and engaging a plunger stop associated with the refill syringe to hold the plunger of the refill syringe in a retracted position relative to a barrel of the refill syringe;
agitating the refill syringe while the predetermined volume of the therapeutic substance resides therein;
disengaging the plunger stop associated with the refill syringe so that the plunger of the refill syringe may move into the barrel of the refill syringe;
attaching an outlet of the filter to the tube;
opening the tubing clamp; and
transferring the predetermined volume of the therapeutic substance from the refill syringe to the reservoir via vacuum pressure in the reservoir.

19. The method of claim 18, further comprising closing the tubing clamp after transferring the predetermined volume of the therapeutic substance from the refill syringe to the reservoir.

20. The method of claim 18, wherein the plunger stop associated with the aspiration syringe is the same as the plunger stop associated with the refill syringe.
